(19) United States
(12) Patent Application Publication
(10) Pub. No.: US 2020/0402610 A1
(43) Pub. Date: Dec. 24, 2020

(54) SYSTEMS AND METHODS FOR DETERMINING GENOME PLOIDY

(71) Applicant: CooperSurgical, Inc., Trumbull, CT (US)

(72) Inventors: John BURKE, Reno, NV (US); Brian RHEES, Reno, NV (US); Joshua David BLAZEK, Houston, TX (US); Michael Jon LARGE, Houston, TX (US)

(21) Appl. No.: 16/907,121

(22) Filed: Jun. 19, 2020

Related U.S. Application Data
(60) Provisional application No. 62/865,122, filed on Jun. 21, 2019.

(51) Int. Cl. G16B 20/10; G16B 20/20; G16B 30/10

(52) U.S. Cl. CPC G16B 20/10; G16B 30/10; G16B 20/20

(57) ABSTRACT

A method for detecting ploidy in an embryo is provided, comprising receiving an embryo sequence data; aligning the received sequence data to a reference genome; identifying a region of interest in the aligned embryo sequence data; identifying single nucleotide polymorphisms (SMPs) in the sequence data by comparing the received sequence data to the aligned reference genome; determining a ploidy score comprising counting the number of observed SNPs in the region of interest; comparing the ploidy score to a predetermined threshold; and identifying the embryo as polyploid if the ploidy score is below the predetermined threshold.

### CROSS-REFERENCE TO RELATED APPLICATIONS

This application claims the benefit of priority to U.S. Provisional Patent Application 62/865,122 filed Jun. 21, 2019, which is incorporated herein by reference in its entirety.

### INCORPORATION BY REFERENCE

The disclosures of any patents, patent applications and publications cited herein are incorporated herein by reference in their entirety.

The embodiments provided herein are generally related to systems and methods for analysis of genomic nucleic acids (genomic DNA) and detection of genetic abnormalities. Included among embodiments provided herein are systems and methods relating to detecting chromosomal abnormalities, such as ploidy (e.g., e.g., haploidy, diploidy and polyploidy), in cells, e.g., an embryo, or organisms.

### BACKGROUND

The low cost of whole genome shotgun (WGS) next generation sequencing (NGS) at very low coverage levels (for example, about 0.1×) allows for relatively inexpensive preimplantation genetic testing of aneuploidy (PGT-A) and unbalanced polyploid states (such as, e.g., 69:XXY, 69:XYY). However, until now, there has been no way to identify/detect non-diploid states, such as 23,X haploid or balanced polyploids like 69:XXX or 92:XXXX using very-low coverage WGS (WGS NGS data). Identification of balanced polyploids is not tenable using existing very low coverage copy number analysis techniques (Shen et al 2016; Liu et al 2015; Park et al 2019) because the ratio of chromosome X to Y abundance is invariant to polyploidy level. SNP microarrays as well as high coverage NGS sequencing (>50×; Weiss et al 2018; >15× Margarido and Heckerman, 2015) can identify 69:XXX, for example, by detecting significant deviation from the expected diploid heterozygous allele ratio of 0.5. However, the analog allele ratio is not usable with low-cost/low-coverage sequencing due to the confounding effects of false homozygosity, sequencing error, and poor statistical power due to low per-locus coverage.

Consequently there is a need for a novel method to detect balanced polyploids using very low coverage WGS NGS data that does not require target enrichment or parental sequence data.

### SUMMARY

Provided herein are methods and systems for analysis of genomic nucleic acids (genomic DNA) and detection and/or identification of genomic features, including, for example, chromosomal abnormalities. In some embodiments, the methods and systems are used in characterizing and/or determining ploidy of a cell(s). In some embodiments, the methods and systems are used in detecting, identifying, determining, inferring and/or distinguishing ploidy (e.g., haploidy, diploidy and polyploidy) and/or euploidy in a cell(s), such as, for example, an embryo (e.g., human), an offspring and/or an organism(s). In some embodiments, the methods and systems are used in detecting, determining and/or identifying balanced polyploidy in a cell(s), e.g., an embryo, such as a preimplantation IVF embryo, offspring or organism.

Methods and systems provided herein include methods of analyzing, assessing, characterizing and/or determining genomes, genomic features and/or genomic nucleic acid (genomic DNA) sequences of a cell or organism. In some embodiments, genomic sequence data used in the methods and systems provided herein are obtained, for example, by nucleic acid sequencing methods, e.g., next generation sequencing (NGS) methods, such as low-coverage and/or low-depth (e.g., low-resolution) sequencing methods. The ability to utilize lower resolution DNA sequencing data obtained from low-coverage and/or low-depth sequencing in methods and systems provided herein provides significant advantages, including, for example, increased efficiency (e.g., allowing multiplex sequencing of a large number of samples) and reduced time and costs. In some embodiments, methods and systems provided herein include detecting, identifying and/or analyzing single nucleotide variation (SNV) in the genome of a cell(s), e.g., an embryo, offspring or organism. In some of such embodiments, the SNV data includes or consists of low resolution sequence information obtained from low-coverage and/or low-depth (e.g., low-resolution) sequencing in methods. In some embodiments, the systems and methods are optimized for using SNV data, such as SNV data generated from low-coverage and/or low-depth (e.g., low-resolution) sequencing methods, to detect, identify, determine, infer and/or distinguish ploidy (e.g., haploidy, diploidy and polyploidy) in a cell(s), such as, for example, an embryo, offspring and/or an organism. In some embodiments, the methods and systems use SNV data, such as SNV data generated from low-coverage and/or low-depth (e.g., low-resolution) sequencing methods, in detecting, inferring, determining, distinguishing and/or identifying balanced polyploidy in a cell(s), e.g., an embryo, such as a preimplantation IVF embryo (e.g., human), offspring or organism.

In accordance with various embodiments, a method is provided for detecting ploidy in an embryo. The method can comprise receiving an embryo sequence data, aligning the received sequence data to a reference genome, identifying a region of interest in the aligned embryo sequence data, identifying single nucleotide polymorphisms (SMPs) in the sequence data by comparing the received sequence data to the aligned reference genome, determining a ploidy score comprising counting the number of observed SNPs in the region of interest, comparing the ploidy score to a predetermined threshold, and identifying the embryo as polyploid if the ploidy score is below the predetermined threshold.

In accordance with various embodiments, a non-transitory computer-readable medium storing computer instructions for detecting ploidy in an embryo is provided. The method can comprise receiving an embryo sequence data, aligning the received sequence data to a reference genome, identifying a region of interest in the aligned embryo sequence data, identifying single nucleotide polymorphisms (SMPs) in the sequence data by comparing the received sequence data to the aligned reference genome, determining a ploidy score comprising counting the number of observed SNPs in the region of interest, comparing the ploidy score to a predetermined threshold, and identifying the embryo as polyploid if the ploidy score is below the predetermined threshold.

In accordance with various embodiments, a system is provided for detecting ploidy in an embryo. The method can comprise a data store for receiving an embryo sequence data, a computing device communicatively connected to the data store, and a display communicatively connected to the computing device and configured to display a report containing the polyploid classification of the embryo. The computing device can comprise an ROI engine configured to align the received sequence data to a reference genome, and identify a region of interest in the aligned embryo sequence data, a SNP identification engine configured to identify single nucleotide polymorphisms (SMPs) in the sequence data by comparing the received sequence data to the aligned reference genome, and a scoring engine configured to determine a polyploid score comprising counting the number of observed SNPs in the region of interest, compare the polyploid score to a predetermined threshold, and identifying the embryo as polyploid if the polyploid score is below the predetermined threshold.

### BRIEF DESCRIPTION OF THE DRAWINGS

FIG. 1 depicts the relationship between the probability of observing an ALT (variant) allele (0% or 100% in homozygotes) in sequence data from sequencing of genomic nucleic acids (genomic DNA) for a euploid (diploid) and aneuploid (trisomic) cell vs sequencing depth, with genotypes having higher ALT frequencies showing higher probabilities of observing an ALT allele, in accordance with various embodiments.

FIG. 2 is an illustration of the difference in the probability of observing an ALT allele in sequence data from sequencing of a euploid genomic DNA sample and the probability of observing an ALT allele in sequence data from sequencing of a trisomy genomic DNA sample, in accordance with various embodiments. Each panel represents variants at different frequencies (0.1, 0.2, 0.3, 0.4), in accordance with various embodiments. Individual plots show the probability of observing an ALT allele given the sequencing depth (constrained to be >=1) for euploid samples (heavy black line) and for trisomy samples (lighter shaded line).

FIG. 3 is a diagrammatic representation of the workflow 300 of an exemplary method for detecting, inferring, identifying, determining and/or distinguishing ploidy, such as polyploidy (e.g., balanced polyploidy) and/or euploidy (e.g., diploidy), in accordance with various embodiments.

FIG. 4 is a representation of the results of an analysis of SNV allele sequence data for embryos of known ploidy used as a training set. The results are shown as a graph of score-polyploid effect as a function of the number of aligned read pairs in the sequencing results. The graph illustrates the training set separation between the ploidy classes (diploid=circles; polyploid=triangles) by sequencing coverage, in accordance with various embodiments.

FIG. 5 is a representation of the results presented in FIG. 4 (illustrating the training set separation between the ploidy classes (diploid and polyploid) by sequencing coverage) after removing the effect of sequencing coverage and other covariates, in accordance with various embodiments.

FIG. 6 is a receiver operating characteristic (ROC) curve evaluated and displayed for the results of the analysis of the training set data (SNV allele sequence data for embryos of known ploidy) shown in FIG. 4 and FIG. 5, in accordance with various embodiments.

FIG. 7 is a representation of the results of an analysis of SNV allele sequence data for embryos of known ploidy used as a training set in. The results are shown as a graph of score-polyploid effect as a function of the number of aligned read pairs in the sequencing results. The graph illustrates the training set separation between the ploidy classes (diploid=circles; polyploid=triangles) by sequencing coverage, in accordance with various embodiments.

FIG. 8 is a representation of the results presented in FIG. 7 (illustrating the training set separation between the ploidy classes (diploid and polyploid) by sequencing coverage) after removing the effect of sequencing coverage and other covariates, in accordance with various embodiments.

FIG. 9 is a histogram illustrating the sensitivities for 2000 iterations of cross validation, in accordance with various embodiments.

FIG. 10 is a schematic diagram of a system for detecting ploidy in an embryo, in accordance with various embodiments.

FIG. 11 is an exemplary flowchart showing a method for detecting ploidy in an embryo, in accordance with various embodiments.

FIG. 12 is a block diagram illustrating a computer system for use in performing methods provided herein, in accordance with various embodiments.

It is to be understood that the figures are not necessarily drawn to scale, nor are the objects in the figures necessarily drawn to scale in relationship to one another. The figures are depictions that are intended to bring clarity and understanding to various embodiments of apparatuses, systems, and methods disclosed herein. Wherever possible, the same reference numbers will be used throughout the drawings to refer to the same or like parts. Moreover, it should be appreciated that the drawings are not intended to limit the scope of the present teachings in any way.

In addition, as the terms “on,” “attached to,” “connected to,” “coupled to,” or similar words are used herein, one element (e.g., a material, a layer, a substrate, etc.) can be “on,” “attached to,” “connected to,” or “coupled to” another element regardless of whether the one element is directly on, attached to, connected to, or coupled to the other element or there are one or more intervening elements between the one element and the other element. In addition, where reference is made to a list of elements (e.g., elements a, b, c), such reference is intended to include any one of the listed elements by itself, any combination of less than all of the listed elements, and/or a combination of all of the listed elements. Section divisions in the specification are for ease of review only and do not limit any combination of elements discussed.

### DETAILED DESCRIPTION

The following description of various embodiments is exemplary and explanatory only and is not to be construed as limiting or restrictive in any way. Other embodiments, features, objects, and advantages of the present teachings will be apparent from the description and accompanying drawings.

Unless defined otherwise, all technical and scientific terms used herein have the same meaning as commonly understood by one of ordinary skill in the art to which this invention belongs. Generally, nomenclatures utilized in connection with, and techniques of, cell and tissue culture, molecular biology, and protein and oligo- or polynucleotide chemistry and hybridization described herein are those well-known and commonly used in the art. Standard techniques are used, for example, for nucleic acid purification and preparation, chemical analysis, recombinant nucleic acid, and oligonucleotide synthesis. Enzymatic reactions and purification techniques are performed according to manufacturer's specifications or as commonly accomplished in the art or as described herein. The techniques and procedures described herein are generally performed according to conventional methods well known in the art and as described in various general and more specific references that are cited and discussed throughout the instant specification. See, e.g., Sambrook et al., Molecular Cloning: A Laboratory Manual (Third ed., Cold Spring Harbor Laboratory Press, Cold Spring Harbor, N.Y. 2000). The nomenclatures utilized in connection with, and the laboratory procedures and techniques described herein are those well-known and commonly used in the art.

A “polynucleotide”, “nucleic acid”, or “oligonucleotide” refers to a linear polymer of nucleosides (including deoxyribonucleosides, ribonucleosides, or analogs thereof) joined by internucleosidic linkages. Typically, a polynucleotide comprises at least three nucleosides. Usually oligonucleotides range in size from a few monomeric units, e.g. 3-4, to several hundreds of monomeric units. Whenever a polynucleotide such as an oligonucleotide is represented by a sequence of letters, such as “ATGCCTG,” it will be understood that the nucleotides are in 5′->3′ order from left to right and that “A” denotes deoxyadenosine, “C” denotes deoxycytidine, “G” denotes deoxyguanosine, and “T” denotes thymidine, unless otherwise noted. The letters A, C, G, and T may be used to refer to the bases themselves, to nucleosides, or to nucleotides comprising the bases, as is standard in the art.

DNA (deoxyribonucleic acid) is a chain of nucleotides containing 4 types of nucleotides; A (adenine), T (thymine), C (cytosine), and G (guanine), and RNA (ribonucleic acid) is comprised of 4 types of nucleotides; A, U (uracil), G, and C. Certain pairs of nucleotides specifically bind to one another in a complementary fashion (called complementary base pairing). That is, adenine (A) pairs with thymine (T) (in the case of RNA, however, adenine (A) pairs with uracil (U)), and cytosine (C) pairs with guanine (G). When a first nucleic acid strand binds to a second nucleic acid strand made up of nucleotides that are complementary to those in the first strand, the two strands bind to form a double strand. As used herein, “nucleic acid sequencing data,” “nucleic acid sequencing information,” “nucleic acid sequence,” “genomic sequence,” “genetic sequence,” or “fragment sequence,” or “nucleic acid sequencing read” denotes any information or data that is indicative of the order of the nucleotide bases (e.g., adenine, guanine, cytosine, and thymine/uracil) in a molecule (e.g., whole genome, whole transcriptome, exome, oligonucleotide, polynucleotide, fragment, etc.) of DNA or RNA. It should be understood that the present teachings contemplate sequence information obtained using all available varieties of techniques, platforms or technologies, including, but not limited to: capillary electrophoresis, microarrays, ligation-based systems, polymerase-based systems, hybridization-based systems, direct or indirect nucleotide identification systems, pyrosequencing, ion- or pH-based detection systems, electronic signature-based systems, etc.

As used herein, the term “cell” is used interchangeably with the term “biological cell.” Non-limiting examples of biological cells include eukaryotic cells, plant cells, animal cells, such as mammalian cells, reptilian cells, avian cells, fish cells or the like, prokaryotic cells, bacterial cells, fungal cells, protozoan cells, or the like, cells dissociated from a tissue, such as muscle, cartilage, fat, skin, liver, lung, neural tissue, and the like, immunological cells, such as T cells, B cells, natural killer cells, macrophages, and the like, embryos (e.g., zygotes), oocytes, ova, sperm cells, hybridomas, cultured cells, cells from a cell line, cancer cells, infected cells, transfected and/or transformed cells, reporter cells and the like. A mammalian cell can be, for example, from a human, mouse, rat, horse, goat, sheep, cow, primate or the like.

A genome is the genetic material of a cell or organism, including animals, such as mammals, e.g., humans, and comprises nucleic acids, i.e., genomic DNA. In humans, total DNA includes, for example, genes, noncoding DNA and mitochondrial DNA. The human genome typically contains 23 pairs of linear chromosomes: 22 pairs of autosomal chromosomes (autosomes) plus the sex-determining X and Y chromosomes. The 23 pairs of chromosomes include one copy from each parent. The DNA that makes up the chromosomes is referred to as chromosomal DNA and is present in the nucleus of human cells (nuclear DNA). Mitochondrial DNA is located in mitochondria as a circular chromosome, is inherited from only the female parent, and is often referred to as the mitochondrial genome as compared to the nuclear genome of DNA located in the nucleus.

As used herein, the phrase “genomic feature” refers to a defined or specified genome element or region. In some instances, the genome element or region can have some annotated structure and/or function (e.g., a chromosome, a gene, protein coding sequence, mRNA, tRNA, rRNA, repeat sequence, inverted repeat, miRNA, siRNA, etc.) or be a genetic/genomic variant (e.g., single nucleotide polymorphism/variant, insertion/deletion sequence, copy number variation, inversion, etc.) which denotes one or more nucleotides, genome regions, genes or a grouping of genome regions or genes (in DNA or RNA) that have undergone changes as referenced against a particular species or sub-populations within a particular species due to, for example, mutations, recombination/crossover or genetic drift.

Ploidy refers to the number of sets (designated as n) of homologous chromosomes in the genome of a cell or organism. For example, a cell or organism having one set of chromosomes is referred to as monoploid. A cell or organism having two sets of homologous chromosomes (2n) is referred to as diploid. Polyploidy is the condition in which a cell(s), e.g., an embryo, offspring or organisms possess more than two complete haploid sets of chromosomes. Haploid refers to cells that have half of the usual complete set of somatic cell chromosomes of an organism. For example, gametes, or reproductive (sex) cells, such as ova and sperm cells in humans, are haploid. Fusion of haploid gametes during fertilization yields a diploid zygote containing one set of homologous chromosomes from the female gamete and one set of homologous chromosomes from the male gamete. A human embryo with a normal number of autosomes (22) and a single sex chromosome pair (XX or XY) is referred to as a euploid embryo. Thus, for humans, the euploid condition is diploid. In various embodiments herein, the phrase “all chromosomes” can include all autosomes and sex chromosomes. In various embodiments herein, the phrase “all chromosomes” does not include sex chromosomes.

The term “allele” refers to alternative forms of a gene. In humans or other diploid organisms, there are two alleles at each genetic locus. Alleles are inherited from each parent: one allele is inherited from the mother and one allele is inherited from the father. A pair of alleles represents the genotype of a gene. If the two alleles at a particular locus are identical, the genotype is referred to as homozygous. If there are differences in the two alleles at a particular locus, the genotype is referred to as heterozygous.

The term “haplotype” refers to a set, or combination, of variations, or polymorphisms, in a chromosome that tend to co-segregate due to proximity in the chromosome. Haplotypes can be described with respect to combinations of variations in a single gene, multiple genes or in sequences between genes. Because of the closeness of the variations in a haplotype, there tends to be little to no recombination or crossover of the locations in which the variations occur and they tend to pass through generations and be inherited together.

As used herein, the phrase “genetic abnormality” refers to a change in a genome relative to a normal, wild-type or reference genome. Generally, genetic abnormalities include chromosomal abnormalities and gene defects. Typically, gene defects include alterations including, but not limited to, single base mutations, substitutions, insertions and deletions and copy number variations. Chromosomal abnormalities include alterations in chromosome number or structure, e.g., duplication and deletion, such as a repeat or loss of a region of a chromosome, inversion and translocation. A common chromosomal abnormality is referred to as aneuploidy which is an abnormal chromosome number due to an extra or missing chromosome. For example, monosomy in a human is an abnormality characterized by a chromosome with a copy loss (only one copy instead of the normal two copies). Trisomy in a human is an abnormality characterized by a chromosome copy gain (three copies instead of the normal two copies). An embryo with an abnormal number of chromosomes is referred to as an aneuploid embryo. Most aneuploidies are of maternal origin and result from errors in segregation during oocyte meiosis. Thus, meiotic aneuploidies will occur in all cells of an embryo. However, mitotic errors are also common in human preimplantation embryos and can result in mitotic aneuploidies and chromosomally mosaic embryos having multiple populations of cells (e.g., some cells being aneuploid and some being euploid). Polyploidy in a human cell is an abnormality in which the cell, e.g., in an embryo, possesses more than two complete sets of chromosomes. Examples of polyploidy include triploidy (3n) and tetraploidy (4n). Polyploidy in humans can occur in several forms that result in having either balanced sex chromosomes or unbalanced sex chromosomes (e.g., detectable by CNV methods). A balanced-sex polyploidy (also referred to as a balanced polyploidy) in humans contains 3 or more complete copies of the haploid genome in which each copy contains only X chromosomes (e.g., 69:XXX or 92:XXXX) or contains an equivalent number of X and Y chromosomes (e.g., 92:XXYY). An unbalanced-sex polyploidy (also referred to as an unbalanced polyploidy) in humans contains 3 or more complete copies of the haploid genome in which at least one copy contains a Y chromosome (e.g., 69:XXY, 69:XYY) and does not contain an equivalent copy number of X and Y chromosomes. Chromosomal abnormalities can have a number of different effects on cells and organisms, including molar pregnancies, miscarriages and genetic disorders and diseases.

In general, genomic variants can be identified using a variety of techniques, including, but not limited to: array-based methods (e.g., DNA microarrays, etc.), real-time/digital/quantitative PCR instrument methods and whole or targeted nucleic acid sequencing systems (e.g., NGS systems, capillary electrophoresis systems, etc.). With nucleic acid sequencing, resolution or coverage can be at one or more levels and is some cases is available at single base resolution.

As used herein, the phrase “pattern of inheritance” refers to the manner and dosage of transmission of a genomic feature, such as, for example, aneuploidy, in the genome of a cell(s), offspring, e.g., an embryo or organism from parent cells or organisms such as diploid cells and organisms. For example, in humans, the offspring, e.g., embryo, receives one gene allele from each parent (one maternal and one paternal) which then make up the two alleles in the diploid cells of the offspring. A pattern of inheritance of a particular allele or genomic feature in an offspring, e.g., an embryo, defines which parent transmitted the genomic feature to the offspring. The parent from whom the genomic feature was transmitted to the offspring or embryo is referred to as the parent of origin. Inheritance can be balanced (expected; equal contribution from each parent) or imbalanced (insufficient or excess). For example, for an embryo possessing Trisomy 21 in which one copy of chromosome 21 was inherited paternally and two copies were inherited maternally, it is said that the parent of origin of aneuploid is maternal. Conversely, for Monsomoy 18, in which an embryo inherited a maternal copy and no paternal copy of chromosome 18, it can be said that the parent of origin for that feature is paternal.

As used herein, “offspring” refers to the product of the union of gametes (e.g., female and male germ cells) and includes, but is not limited to, e.g., a blastomere, a zygote, an embryo, fetus, neonate or child. Offspring DNA can be obtained from any source, including, for example, a blastomere biopsy, a trophectoderm biopsy, an inner cell mass biopsy, a blastocoel biopsy, embryo spent media, cfDNA, products of conception, chorionic villus samples and/or amniocentesis.

As used herein, “parent” or “genetic parent” refers to a contributor of a gamete to an offspring and includes, for example, egg and sperm donors so long as the gamete DNA originates from the donor.

The phrase “mosaic embryo” denotes embryos containing two or more cytogenetically distinct cell lines. For example, a mosaic embryo can contain cell lines with different types of aneuploidy or a mixture of euploid and genetically abnormal cells containing DNA with genetic variants that may be deleterious to the viability of the embryo during pregnancy.

The phrase “next generation sequencing” (NGS) refers to sequencing technologies having increased throughput as compared to traditional Sanger- and capillary electrophoresis-based approaches, for example with the ability to generate hundreds of thousands of relatively small sequence reads at a time. Some examples of next generation sequencing techniques include, but are not limited to, sequencing by synthesis, sequencing by ligation, and sequencing by hybridization. More specifically, the MISEQ, HISEQ and NEXTSEQ Systems of IIlumina and the Personal Genome Machine (PGM), Ion Torrent, and SOLiD Sequencing System of Life Technologies Corp, provide massively parallel sequencing of whole or targeted genomes. The SOLiD System and associated workflows, protocols, chemistries, etc. are described in more detail in PCT Publication No. WO 2006/084132, entitled “Reagents, Methods, and Libraries for Bead-Based Sequencing,” international filing date Feb. 1, 2006, U.S. patent application Ser. No. 12/873,190, entitled “Low-Volume Sequencing System and Method of Use,” filed on Aug. 31, 2010, and U.S. patent application Ser. No. 12/873,132, entitled “Fast-Indexing Filter Wheel and Method of Use,” filed on Aug. 31, 2010, the entirety of each of these applications being incorporated herein by reference thereto.

The phrase “sequencing run” refers to any step or portion of a sequencing process performed to determine some information relating to at least one biomolecule (e.g., nucleic acid molecule).

The term “read” with reference to nucleic acid sequencing refers to the sequence of nucleotides determined for a nucleic acid fragment that has been subjected to sequencing, such as, for example, NGS. Reads can be any a sequence of any number of nucleotides which defines the read length.

The phrase “sequencing coverage” or “sequence coverage,” used interchangeably herein, generally refers to the relation between sequence reads and a reference, such as, for example, the whole genome of cells or organisms, one locus in a genome or one nucleotide position in the genome. Coverage can be described in several forms (see, e.g., Sims et al. (2014) Nature Reviews Genetics 15:121-132). For example, coverage can refer to how much of the genome is being sequenced at the base pair level and can be calculated as NL/G in which N is the number of reads, L is the average read length, and G is the length, or number of bases, of the genome (the reference). For example, if a reference genome is 1000 Mbp and 100 million reads of an average length of 100 bp are sequenced, the coverage would be 10×. Such coverage can be expressed as a “fold” such as 1×, 2×, 3×, etc. (or 1, 2, 3, etc. times coverage). Coverage can also refer to the redundancy of sequencing relative to a reference nucleic acid to describe how often a reference sequence is covered by reads, e.g., the number of times a single base at any given locus is read during sequencing. Thus, there may be some bases which are not covered and have a depth of 0 and some bases that are covered and have a depth of anywhere between, for example, 1 and 50. Redundancy of coverage provides an indication of the reliability of the sequence data and is also referred to as coverage depth. Redundancy of coverage can be described with respect to “raw” reads that have not been aligned to a reference or to aligned (e.g., mapped) reads. Coverage can also be considered in terms of the percentage of a reference (e.g., a genome) covered by reads. For example, if a reference genome is 10 Mbp and the sequence read data maps to 8 Mbp of the reference, the percentage of coverage would be 80%. Sequence coverage can also be described in terms of breadth of coverage which refers to the percentage of bases of a reference that are sequenced a given number of times at a certain depth.

As used herein, the phrase “low coverage” with respect to nucleic acid sequencing refers to sequencing coverage of less than about 10×, or about 0.001× to about 10×, or about 0.002× to about 0.2×, or about 0.01× to about 0.05×.

As used herein, the phrase “low depth” with respect to nucleic acid sequencing refers to an average genome-wide sequencing depth of less than about 20× or less than about 10×, or about 0.1× to about 10×, or about 0.2× to about 5×, or about 0.5× to about 2×.

The term “resolution” with reference to genomic sequence nucleic acid sequence refers to the quality, or accuracy, and extent of the genomic nucleic acid sequence (e.g., DNA sequence of the entire genome or a particular region or locus of the genome) obtained through nucleic acid sequencing of a cell(s), e.g., an embryo, or organism. The resolution of genomic nucleic acid sequence is primarily determined by the coverage and depth of the sequencing process and involves consideration of the number of unique bases that are read during sequencing and the number of times any one base is read during sequencing. The phrases “low resolution sequence” or “low resolution sequence data” or “sparse sequence data,” which are used interchangeably herein, with reference to genomic nucleic acid sequence (genomic DNA) of a cell(s), e.g., an embryo, offspring or organism, refer to the nucleotide base sequence information of genomic nucleic acid (genomic DNA) that is obtained through low-coverage and low-depth sequencing methods.

All publications mentioned herein are incorporated herein by reference for the purpose of describing and disclosing devices, compositions, formulations and methodologies which are described in the publication and which might be used in connection with the present disclosure.

As used herein, the terms “comprise”, “comprises”, “comprising”, “contain”, “contains”, “containing”, “have”, “having” “include”, “includes”, and “including” and their variants are not intended to be limiting, are inclusive or open-ended and do not exclude additional, unrecited additives, components, integers, elements or method steps. For example, a process, method, system, composition, kit, or apparatus that comprises a list of features is not necessarily limited only to those features but may include other features not expressly listed or inherent to such process, method, system, composition, kit, or apparatus.

The practice of the present subject matter may employ, unless otherwise indicated, conventional techniques and descriptions of organic chemistry, molecular biology (including recombinant techniques), cell biology, and biochemistry, which are within the skill of the art.

### Detection/Determination of Ploidy Level

Polyploidy is a condition in which cells, e.g., an embryo, or organisms possess more than two complete haploid sets of chromosomes. In a human fetus, polyploidy is a highly lethal abnormality. Of all first trimester miscarriages with confirmed aneuploidy (spontaneous conception and IVF), 10-15% are the result of polyploidy. Examples of polyploidy include triploidy (3n) and tetraploidy (4n). Triploidy is estimated to affect 1-3% of IVF embryos and can lead to molar pregnancies and miscarriages. The extra set of chromosomes that occurs in triploidy can be maternal (digynic) or paternal (diandric) in origin. Polyploidy in humans can described as “balanced” or “unbalanced.” A balanced-sex polyploidy (also referred to as a balanced polyploidy) in humans contains 3 or more complete copies of the haploid genome in which each copy contains only X chromosomes (e.g., 69:XXX or 92:XXXX) or contains an equivalent number of X and Y chromosomes (e.g., 92:XXYY). An unbalanced-sex polyploidy (also referred to as an unbalanced polyploidy) in humans contains 3 or more complete copies of the haploid genome in which at least one copy contains a Y chromosome (e.g., 69:XXY, 69:XYY) and does not contain an equivalent copy number of X and Y chromosomes. Polyploidy is distinguished from aneuploidies, such as trisomy, which, although is characterized by an aberrant number of chromosomes, does not involve one or more additional complete sets of chromosomes. Thus, trisomy occurs in a human when an extra copy of one chromosome is present in the genome instead of an extra copy of each chromosome as is the case in triploidy.

Detection of ploidy such as, polyploidy for example, presents challenges when using nucleic acid sequencing-based methods for analysis of chromosomal copy number variations. For example, in using sequence read data to detect an extra chromosome in the case of trisomy, it is possible to compare the numbers of reads for any particular chromosome to those of a reference chromosome and identify disproportionalities as indicative of trisomy. However, in some cases of triploidy, such as balanced triploidy, a reference chromosome is not available since all chromosomes are present in equal dosage (e.g., trisomic) and the relative ratio of sequence reads for all chromosomes is the same as it would be for a euploid cell or organism. Some methods leverage sex chromosome ratios relative to autosomes to infer incidence of male triploidy, but female triploidy (as well as 23,X monoploidy) cannot be detected in this manner. When DNA is sequenced at great depth (e.g., high-resolution sequencing), accurate SNP quantification, alone or in conjunction with other methods, can be utilized to identify triploidy and overcome false homozygosity and sequencing errors to detect balanced triploidy. However, such methods are associated with relatively high costs, longer run and analysis times and lower throughput and efficiencies as compared to low-coverage and/or low-depth, e.g., low-resolution, sequencing methods. The low-resolution sequence data provided by low-coverage and/or low-depth, e.g., low-resolution, sequencing methods is sparse, with missing data points for sequence information that is needed to attempt to detect balanced polyploidy. Additionally, DNA samples require processing, including, for example, fragmentation, amplification and adapter ligation prior to sequencing via NGS. Manipulations of the nucleic acids in such processing may introduce artifacts (e.g., GC bias associated with polymerase chain reaction (PCR) amplification), into the amplified sequences and limit the size of sequence reads. Next generation sequencing (NGS) methods and systems are thus associated with error rates that may differ between systems. Additionally, software used in conjunction with identifying bases in a sequence read (e.g., base-calling) can affect the accuracy of sequence data from NGS sequencing. These artifacts, variations in coverage and errors that can occur in NGS have a more pronounced effect in interpretation of low-coverage sequencing data as compared to high-coverage sequencing data.

Provided herein are improved, efficient, rapid, and cost-effective methods and systems for detecting, identifying and/or distinguishing ploidy, such as polyploidy (e.g., balanced polyploidy) and/or euploidy (e.g., diploidy) in a cell(s), such as, for example, an embryo, and/or an organism. In some embodiments of methods and systems provided herein, relatively low-coverage and/or low-depth, e.g., low-resolution, sequence data are used to detect, distinguish, infer and/or identify ploidy, such as euploidy and/or polyploidy, e.g., balanced polyploidy, in a cell(s), e.g., cells of an embryo, offspring or organism. In some such embodiments, the systems and methods are used to detect, distinguish, infer and/or identify triploidy or tetraploidy, such as balanced triploidy or tetraploidy. In some such embodiments, the methods and systems are used to detect, distinguish, infer and/or identify triploidy or tetraploidy, such as balanced triploidy or tetraploidy, in an embryo, including, for example, an embryo (e.g., a mammalian embryo such as a human embryo) generated through IVF, prior to implantation. In some embodiments, the methods, and systems incorporating the methods, use low-resolution nucleic acid sequence data obtained from low-coverage and low-depth whole genome sequencing of nucleic acid (DNA) samples of the total or complete genomic DNA of a cell(s) (e.g., the total nuclear or chromosomal nucleic acids and/or total DNA of a cell) as opposed to sequencing of only pre-determined specific targeted regions of a genome as would be the case in sequencing of a collection of nucleic acids obtained from targeted nucleic acid amplification of genomic nucleic acids. Use of sequence data from total or complete genomic nucleic acids (e.g., the total nuclear or chromosomal nucleic acids) enables a global assessment of genomic sequences in detecting, identifying and/or distinguishing ploidy, such as polyploidy (e.g., balanced polyploidy) and/or euploidy (e.g., diploidy) in some embodiment of methods provided herein. Such methods involving global assessment of genomic nucleic acid sequences, which are not reliant on sex chromosome/autosomal chromosome ratios for inferring polyploidy, allow for the detection of female (XXX) polyploidy as well as detection and/or confirmation of male (XXY) polyploidy (and haploidy as well). In embodiments that use sequence data obtained from sequencing of nucleic acid samples of the total or complete genomic nucleic acid (e.g., the total nuclear or chromosomal nucleic acids) as opposed to sequencing of only pre-determined specific targeted regions of a genome, such embodiments of the methods and systems provided herein are able to avoid the decreased efficiency and increased preparation time associated with preparation of targeted nucleic acid samples for sequencing. Furthermore, targeted amplification involves additional nucleic acid manipulations that can introduce errors, artifacts and bias into the sequencing data and excludes sequence data from all other, non-targeted regions of the genome that may be more informative in evaluating ploidy and detecting polyploidy. Methods and systems provided herein for detecting, identifying and/or distinguishing ploidy, such as polyploidy (e.g., balanced polyploidy) and/or euploidy (e.g., diploidy) in a cell(s), such as, for example, an embryo, and/or an organism also do not require, and in some embodiments are performed, without nucleic acid sequence information from sequencing of nucleic acids of one or both parents. This provides further advantages of increased efficiency, cost-effectiveness and reduced analysis and computation times of the methods and systems provided herein as compared to other methods of detecting and/or identifying polyploidy, such as balanced polyploidy.

### Nucleic Acid Sequence Data Generation

Some embodiments of the methods and systems provided herein for detecting, identifying, inferring and/or distinguishing ploidy, such as polyploidy (e.g., balanced polyploidy) and/or euploidy (e.g., diploidy) and/or haploidy in a cell(s), such as, for example, an embryo, offspring and/or an organism include analysis of nucleotide sequences of the genome of cells and/or organisms. Nucleic acid sequence data can be obtained using a variety of methods described herein and/or know in the art. In one example, sequences of genomic nucleic acid of cells, for example cells of an embryo, may be obtained from next-generation sequencing (NGS) of DNA samples extracted from the cells. NGS, also known as second-generation sequencing, is based on high-throughput, massively parallel sequencing technologies that involve sequencing of millions of nucleotides generated by nucleic acid amplification of samples of DNA (e.g., extracted from embryos) in parallel (see, e.g., Kulski (2016) “Next-Generation Sequencing—An Overview of the History, Tools and ‘Omic’ Applications,” in Next Generation Sequencing—Advances, Applications and Challenges, J. Kulski ed., London: Intech Open, pages 3-60). Nucleic acid samples to be sequenced by NGS are obtained in a variety of ways, depending on the source of the sample. For example, human nucleic acids may readily be obtained via cheek brush swabs to collect cells from which nucleic acids are then extracted. In order to obtain optimum amounts of DNA for sequencing from embryos (for example, for pre-implantation genetic screening), cells (e.g., 5-7 cells) commonly are collected through trophectoderm biopsy during the blastocyst stage.

Artifacts, variations in coverage and errors that can occur in NGS also present challenges in the analysis of sequence data to accurately evaluate ploidy. Such artifacts and limitations can make it difficult to sequence and map long repetitive regions of a genome and identify polymorphic alleles and aneuploidy in genomes. For example, because about 40% of the human genome is comprised of repeat DNA elements, shorter single reads of identical sequence that align to a repeat element in a reference genome often cannot be accurately mapped to a particular region of the genome. One way to address and possibly reduce some of the effects of errors and/or incompleteness in sequence determination is by incorporating paired-end sequencing techniques into the sequencing method. Paired-end sequencing increases accuracy in placement of sequence reads, e.g., in long repetitive regions, when mapping sequences to a genome or reference, and increases resolution of structural rearrangements such as gene deletions, insertions and inversions. For example, in some embodiments of methods provided herein, use of data obtained from paired-end NGS of nucleic acids from embryos increased read mapping by an average of 15%. Paired-end sequencing methods are known in the art and/or described herein and involve determining the sequence of a nucleic acid fragment in both directions (i.e., one read from one end of the fragment and a second read from the opposite end of the fragment). Paired-end sequencing also effectively increases sequencing coverage redundancy by doubling the number of reads and particularly increases coverage in difficult genomic regions.

### Nucleic Acid Sequence Mapping

In some embodiments of the methods and systems provided herein for detecting, identifying and/or distinguishing ploidy, such as polyploidy (e.g., balanced polyploidy) and/or euploidy (e.g., diploidy) in a cell(s), such as, for example, an embryo, and/or an organism, the sequences of nucleic acids obtained from cells, e.g., embryo cells, or organisms are used to reconstruct the genome (or portions of it) of the cells/organisms using methods of genomic mapping. Typically, genomic mapping involves matching sequences to a reference genome (e.g., a human genome) in a process referred to as alignment. Examples of human reference genomes that may be used in mapping processes include releases from the Genome Reference Consortium such as GRCh37 (hg19) released in 2009 and GRCh38 (hg38) released in 2013 (see, e.g., https://genome.ucsc.edu/cgi-bin/hgGateway?db=hg19

https://www.ncbi.nlm.nih.gov/assembly/GCF_000001405.39). Through alignment, sequence reads are assigned to genomic loci typically using computer programs to carry out the matching of sequences. Numerous alignment programs are publicly available and include Bowtie (see, e.g., http://bowtie-bio.sourceforge.net/manual.shtml) and BWA (see, e.g., http://bio-bwa.sourceforge.net/). Sequences that have been processed (for example to remove PCR duplicates and low-quality sequences) and matched to a locus are often referred to as aligned sequences or aligned reads.

In mapping of sequence reads to a genomic reference, it is possible to identify sequence nucleotide variants (SNV). Single nucleotide variants are the result of variation in the genome at a single nucleotide position. Several different NGS analysis programs for SNV detection (e.g., variant calling software) are publicly available, known in the art and/or described herein (e.g., including but not limited to GATK (see, e.g., https://gatk.broadinstitute.org/) and deepvariant (see, e.g., Poplin et al (2018) Nature Biotech. 36:983-987). After alignment, the bcftools software (open source) is used to generate a pileup of all bases identified with a minimum coverage (e.g., 1) and minimum depth (e.g., 1) and generate a genotype call from the bam file generated during alignment. Detection and identification of genomic features, such as chromosomal abnormalities, e.g., polyploidies, through genome mapping of sequences from sample nucleic acids of cells or organisms presents particular challenges, particularly when sequence data is obtained from low-coverage sequencing methods. For example, deciphering signal from noise in sparse sequence data is more challenging than it is for high-resolution sequence obtained from high-coverage sequencing. The major challenges in this approach are derived from the concept that NGS methods are prone to introducing errors into the sequencing read during read generation. With error rates anywhere between 1:100 and 1:10,000, depending on the sequencing platform methodology, identifying the difference between a variant and sequencing error at low-coverage and/or low-depth sequencing provides a unique and difficult informatics challenge. Computer programs and systems are known in the art and/or described herein for increasing the ease and/or accuracy of interpretation of sequence data in identifying certain genomic features. For example, systems and methods for automated detection of chromosomal abnormalities including segmental duplications/deletions, mosaic features, aneuploidy and polyploidy with unbalanced sex chromosomes are described in U.S. Patent Application Publication No. 2020/0111573 which is incorporated in its entirety by reference herein. Such methods can include de-noising/normalization (to de-noise raw sequence reads and normalize genomic sequence information to correct for locus effects) and machine learning and artificial intelligence to interpret (or decode) locus scores into karyograms. For example, after sequencing is completed, the raw sequence data is demultiplexed (attributed to a given sample), reads are aligned to a reference genome such as, e.g., HG19, and the total number of reads in each 1-million base pair bin is counted. This data is normalized based on GC content and depth and tested against a baseline generated from samples of known outcome. Statistical deviations from a copy number of 2 are then reported (if present, if not=euploid) as aneuploidy. Using this method, meiotic aneuploidy and mitotic aneuploidy can be distinguished from each other based on the CNV (chromosomal, or portion thereof, copy number variation) metric. Based on the deviations from normal, a karyotype is generated with the total number of chromosomes present, any aneuploidies present, and the mosaic level (if applicable) of those aneuploidies.

### Single Nucleotide Variation in Euploidy and Polyploidy (e.g., Non-Diploid Polyploidy)

In methods and systems provided herein for detecting, identifying, determining, inferring and/or distinguishing ploidy, such as polyploidy (e.g., balanced polyploidy, non-diploidy polyploidy) and/or euploidy (e.g., diploidy) and/or haploidy in a cell(s), such as, for example, an embryo, offspring and/or an organism, SNV sequence information from one or more, or a plurality, of cells, e.g., cells of an embryo, is used in the analysis of ploidy. In some embodiments, the SNV sequence is low-resolution sequence data obtained from low-coverage and/or low depth, e.g., low-resolution, sequencing of genomic nucleic acids (genomic DNA) of the cell(s). In some embodiments of the methods and systems for detecting, inferring, determining, identifying and/or distinguishing ploidy, such as polyploidy (e.g., balanced polyploidy, non-diploid polyploidy), the SNV sequence information is obtained from whole genome sequencing, e.g., of complete genomic DNA samples (e.g., total nuclear or chromosomal nucleic acid samples). In some such embodiments, the SNV sequence information is low-resolution sequence data obtained from low-coverage and low-depth whole genome sequencing. If more than 1% of a population does not carry the same nucleotide at a specific position in the genome, the SNV is often referred to as a single nucleotide polymorphism (SNP). A SNV is typically a more generic term for less well-characterized loci. There are about 10 million or more SNPs located throughout the human genome, on average every 200 bp. Although some SNPs may be associated with traits or disorders, most have no known function. No two individuals (except identical twins) have the same pattern of SNPs which exist as major and minor isoforms within a given population. SNV and SNP are used interchangeably herein.

In using SNV sequence information from a cell(s), e.g., of an embryo, or offspring, methods and systems provided herein include determining the number of SNV alleles present in sequence data from sequencing of total DNA (e.g., total DNA or genomic DNA) and the incidence of reference and/or alternate alleles detected as a function of the total number of SNV alleles. This information provides an actual observed alternate allele determination. A reference (REF) allele in the sequence information refers to a form of a particular nucleotide sequence in the genome that contains a reference nucleobase at a variant position in the sequence. The reference nucleobase is the nucleobase (A, G, T or C) that is in the variant position in the reference genome to which the sequence reads were aligned in mapping the of the SNVs used in the methods. An alternate (ALT) allele in the sequence information refers to a form of a particular nucleotide sequence in the genome that contains a nucleobase that is different from the reference nucleobase at the variant position in the sequence. In a human euploid (i.e., diploid) embryo, one set of chromosomes is maternal in origin and the other is paternal in origin and the overall SNV pattern (the nucleobase identities at each SNV position in the genome for all variant positions) of the two separate sets of chromosomes will differ (i.e., there are two different SNV patterns and the embryo contains one “dose” of each pattern). Within each overall SNV pattern, there are individual variant positions that have the same nucleobase (e.g., both REF nucleobases or both ALT nucleobases) in each set of chromosomes, and individual variant positions that have different nucleobases in the separate sets of chromosomes (one having a REF nucleobase and the other having an ALT nucleobase). In a human triploid embryo, two sets of chromosomes originate from one parent and thus exhibit SNV patterns consistent with said parent, and the third set of chromosomes originates from the other parent and has a different SNV pattern. Therefore the dose of one parental SNV pattern is twice that of the other SNV pattern in triploidy. Thus, in this generalized description for purposes of illustration dosage imbalance, in the case of triploidy in a genome of a human cell, for a particular SNV-containing allele that differs between the two different sets of chromosomes, there could be a different amount, e.g., twice the amount, of sequence available for one form of the allele (e.g., a REF allele) than there is for a different form of the allele (e.g., an ALT allele). In contrast, in this generalized illustration, in a euploid (i.e., diploid) human cell, for a particular SNV-containing allele that differs between the two different sets of chromosomes, the amount of sequence available for one form of the allele (e.g., a REF allele) can be more equivalent to the amount of sequence available for the different form of the allele (e.g., an ALT allele) in respect to alleles that are heterozygous. There is a greater possibility that sequence for one allele of a variant from one set of chromosomes may be missing in low-resolution sequence data obtained from low-coverage sequencing of nucleic acids from a euploid human embryo, than in high-resolution sequence data obtained from high-coverage sequencing. This possibility is further increased in the case of low-resolution sequence data for genomic nucleic acids from a polyploid e.g., triploid, human embryo, particularly in the case of balanced polyploidy.

As described and established herein, theoretical stochastic behavior of the observed single nucleotide variation (SNV) rate (the function that is likelihood of observation vs. prevalence in a sample) differs measurably between diploid and triploid states due to interactions between genotype occurrence probability, minor-allele frequency, sequencing and ploidy state. In some embodiments of the methods and systems provided herein, the difference in SNV rates of haploid, euploid and/or polyploid genomes is included in determining an inference of ploidy, e.g., euploidy or polyploidy, such as balanced polyploidy using low-to-very low coverage genome sequencing (e.g., whole genome sequencing). In such embodiments, a statistic developed based on SNV rate is used in the methods and systems that is able to detect and/or identify polyploidy with around 90% sensitivity and specificity from low-resolution sequence data obtained in low-coverage (e.g., 0.1× coverage) and/or low-depth NGS sequencing.

### Differences in the Probabilities of Observing an ALT Allele in Euploid and Polyploid Genomes

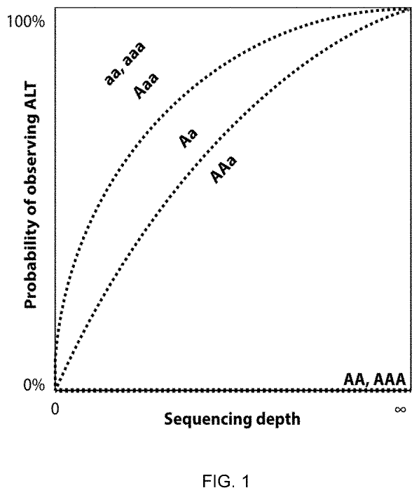

Intuitively, the probability of detecting an allele in sequence reads from genomic DNA sequencing depends, in part, on the allele frequency in a test genomic DNA sample due to underlying genotype. In addition, the probability of detecting an allele depends on sequencing depth (e.g., redundancy of sequencing). FIG. 1 depicts the relationship between the probability of observing an ALT (i.e., variant allele) allele (“a” in this example in which “A” is considered the REF allele) in sequence data from sequencing of genomic DNA for a euploid (diploid) and aneuploid (trisomic) cell vs. sequencing depth. The boundary cases for allele frequencies are homozygote samples (frequency 0% or 100%). The boundary cases for sequencing depth are zero or infinite (no reads with that allele or infinity reads with that allele).

For boundary conditions, the probability of observing the ALT allele is identical for euploid or aneuploid heterozygote samples. In between the two extremes, the expectation is that samples with higher ALT frequencies are more likely to report ALT alleles (see FIG. 1 and Table 1).

TABLE 1
Frequency (or probability) of reference and alternate
alleles given sample genotype

| Genotype | Type | Pr(A) | Pr(a) |
|---|---|---|---|
| aa or aaa | Homozygous euploid or aneuploid | 0 | 1 |
| | sample (variant) | | |
| Aaa | Aneuploid heterozygote | ⅓ | ⅔ |
| Aa | Euploid heterozygote | ½ | ½ |
| AAa | Aneuploid heterozygote | ⅔ | ⅓ |
| AA or AAA | Homozygous euploid or aneuploid | 1 | 0 |
| | sample (reference) | | |

A = Reference or REF allele;
a = Alternate, variant or ALT allele

However, samples of genomic nucleic acids from aneuploid cells, in aggregate, will show different ALT allele sequence counts than samples of genomic nucleic acids from euploid cells, as the dosage imbalance will skew the net actual incidence of alternate vs. reference alleles. To calculate the probability of observing variant alleles (i.e., observing both whether the variant allele is in the sequence data and whether it was in the sample) in the euploid and trisomy cases, consider the equation 1 below:

Pr(ALT|k)=Σ_{G}Pr(G)P(ALT|G,k) (1)

At any given site, therefore, the probability of observing an ALT allele given a sequence depth of k [Pr(ALT|k] can be equal to the (a) probability of observing the ALT allele for any given genotype G [P(ALT|G, k)] (e.g., in connection with the relationship between the number of reads for an ALT allele and the number of instances of the ALT allele in the genomic DNA) adjusted by the (b) probability of the genotype [Pr(G)]. Further discussion of (a) and (b) terms follows below.

P(ALT|G,k)

As described above, the probability of observing a non-reference or ALT allele at a given site can depend on two factors: (1) the frequency of the ALT allele at the site given the genotype (e.g. a euploid heterozygous subject can have an expected ALT frequency of 0.5), and (2) the depth of sequencing. Regarding (2), very deep sequencing, for example, can ensure that an ALT allele will be observed when present, whereas shallow sequencing may miss the ALT allele (“false homozygosity”).

In summary, this can be viewed as a type of binomial probability with the reference (REF) allele probability p and with sequencing count k alleles at the site. As such, the probability of detecting an ALT allele [P(ALT|G,k] (i.e., probability of detecting an allele in the sequence data) can be 1 minus the probability of detecting the reference allele, i.e.:

P(ALT|G,k)=1−p^{k} (2)

Note that the probability p of the reference allele is the frequency of the reference allele in the genotype. For example, for a euploid heterozygote (Aa), p=0.5. For example, if a site were sequenced 10 times and the underlying site was euploid heterozygous, then the probability of not observing an ALT in all 10 reads is 0.5¹⁰, and therefore the probability of observing an ALT is 1−0.5¹⁰.

### Probability of Genotype at a Given Site [Pr(G)]

For euploidy, the assumption may be an independence of chromosomes inherited from each parent, such that the probability of a given genotype, under Hardy-Weinberg equilibrium (HWE), is as follows:

Pr(AA)=Pr(A)²

Pr(Aa)=2Pr(A)Pr(a)

Pr(aa)=Pr(a)²

For euploidy, one can calculate the conditional probabilities of the embryo genotypes given the parental genotypes (see Table 2).

TABLE 2
Parental genotypes, their population frequencies, and
the conditional probabilities of
euploid embryo genotypes, given normal meiosis.

| | | | Conditional probability of | | |
|---|---|---|---|---|---|
| Genotypes | | Pop | embryo genotype | | |
| Maternal | Paternal | Freq | AA | Aa | aa |
| AA | AA | A⁴ | 1 | | |
| AA | Aa | 2A³a | 0.5 | 0.5 | |
| AA | aa | A²a² | | 1 | |
| Aa | AA | 2A³a | 0.5 | 0.5 | |
| Aa | Aa | 4A²a² | 0.25 | 0.5 | 0.25 |
| Aa | aa | 2Ar³ | | 0.5 | 0.5 |
| aa | AA | A²a² | | 1 | |
| aa | Aa | 2Ar³ | | 0.5 | 0.5 |
| aa | aa | a⁴ | | | 1 |
| Total | | 1 | A⁴ + 2A³a + | 2A³a + 4A²a² + | A²a² + |
| | | | A²a² | 2Aa³ | 2Aa³ + a⁴ |

Allele frequencies A = Pr(A), a = Pr(a), with A + a = 1
Conditional probabilities of disjunction (d) m = Pr(dₘ | d), and p = Pr(dₚ | d), with m + p = 1

The probability of the trisomy embryo genotypes can be calculated using the assumption of independence of parental chromosomes, while allowing for parent-specific nondisjunction (m and p), i.e.

m=Pr(dₘ|d), and (3)

p=Pr(dₚ|d) (4)

where m is the probability that a given nondisjunction occurred in the maternal gamete, and p is the probability that the nondisjunction occurred in the paternal gamete. As these are conditional, m+p=1.

For trisomy, the conditional probabilities of the embryo genotypes can be calculated given the parental genotypes and the conditional probability of nondisjunction (see Table 3).

TABLE 3
Parental genotypes, their population frequencies, and the conditional probabilities
of embryo genotypes, given a meiotic nondisjunction event.

| Genotypes | | | Conditional probability of embryo genotype | | | |
|---|---|---|---|---|---|---|
| Maternal | Paternal | Pop Freq | AAA | AAa | Aaa | aaa |
| AA | AA | A⁴ | 1 | | | |
| AA | Aa | 2A³a | 0.5 | 0.5m | 0.5p | |
| AA | aa | A²a² | | m | p | |
| Aa | AA | 2A³a | 0.5 | 0.5p | 0.5m | |
| Aa | Aa | 4A²a² | 0.25 | 0.25 | 0.25 | 0.25 |
| Aa | aa | 2Aa³ | | 0.5m | 0.5p | 0.5 |
| aa | AA | A²a² | | p | m | |
| aa | Aa | 2Aa³ | | 0.5p | 0.5m | 0.5 |
| aa | aa | a⁴ | | | | 1 |
| Total | | 1 | A⁴ + 2A³a + | A³a + 2A²a² + | A³a + 2A²a² + | A²a² + 2Aa³ + |
| | | | A²a² | Aa³ | Aa³ | a⁴ |

Allele frequencies A = Pr(A), a = Pr(a), with A + a = 1
Conditional probabilities of disjunction (d) m = Pr(dₘ|d), and p = Pr(dₚ|d), with m + p = 1

Regarding Tables 2 and 3 above, it should be noted that (a) unconditional probabilities of observing homozygotes (either AA vs AAA or aa vs aaa) can be identical for euploid and trisomy embryo samples, and (b) unconditional probabilities for trisomy heterozygotes (AAa or Aaa) can be identical and sum to the probability of a heterozyote for a euploid sample (Aa).

Equation 1, discussed above, can be expanded for the euploid case as follows:

P(ALT|k)=(A⁴+2A³a+A²a²)(1−1^{k})+(2A³a+4A²a²+2Aa³)(1−0.5^{k})+(A²a²+2Aa³+a⁴)(1−0^{k}) (5)

P(ALT|k)=(2A³a+4A²a²+2A^{a}^{³})(1−0.5^{k})+(A²a²+2Aa³+a⁴) (6)

Equation 1, discussed above, can also be expanded for the trisomy case as follows:

P(ALT|k)=(A⁴+2A³a+A²a²)(1−1k)+(A³a+2A²a²+Aa³)(1−(⅔)^{k})+(A³a+2A²a²+Aa³)(1−(⅓)^{k})+(A²a²+2Aa³+a⁴)(1−0^{k}) (7)

P(ALT|k)=(A³a+2A²a²+Aa³)(1−(⅔)^{k})+(A³a+2A²a²+Aa³)(1−(⅓)^{k})+(A²a²+2Aa³+a⁴) (8)

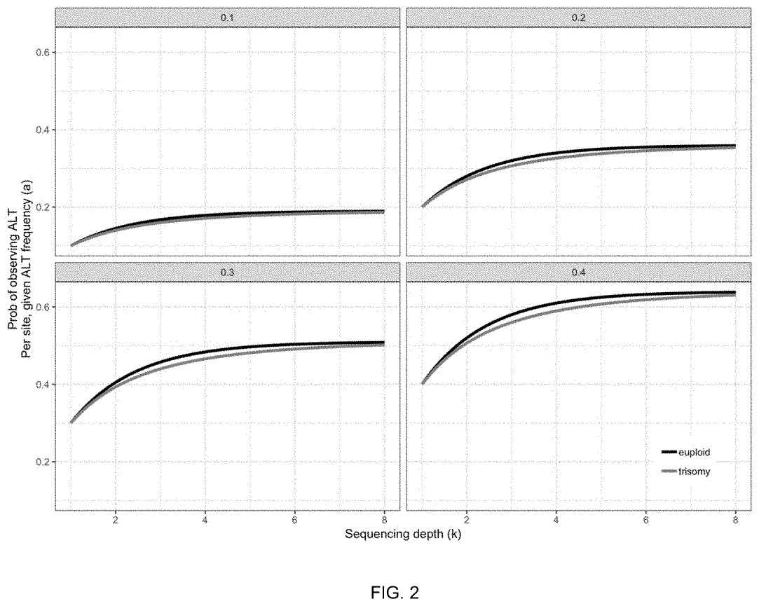

As such, the probabilities of observed variants under the two cases (for euploid embryos and triploid embryos) can be compared, as shown in FIG. 2. The graphs in FIG. 2 illustrate the difference in the probability of observing an ALT allele in sequence data from sequencing of a euploid genomic nucleic acid sample (heavy black curves) and the probability of observing an ALT allele in sequence data from sequencing of a trisomy genomic nucleic acid sample (lighter shaded curves). The probabilities are shown as a function of sequencing depth (constrained to be >=1×). Each panel represents the probabilities at different frequencies (prevalence in the sample) (0.1, 0.2, 0.3, 0.4). As shown in FIG. 2, the differences in the probabilities of observing an ALT allele in sequence data from sequencing of a euploid genomic nucleic acid sample and the probability of observing an ALT allele in sequence data from sequencing of a trisomy genomic nucleic acid sample diminish for larger k values (i.e., increased sequencing depth). Moreover, the extent of the difference in probability of observing an ALT difference can vary based on the genotype, which can depend on the population allele frequency.

### Methods and Systems for Detecting, Identifying, Determining and/or Distinguishing Ploidy

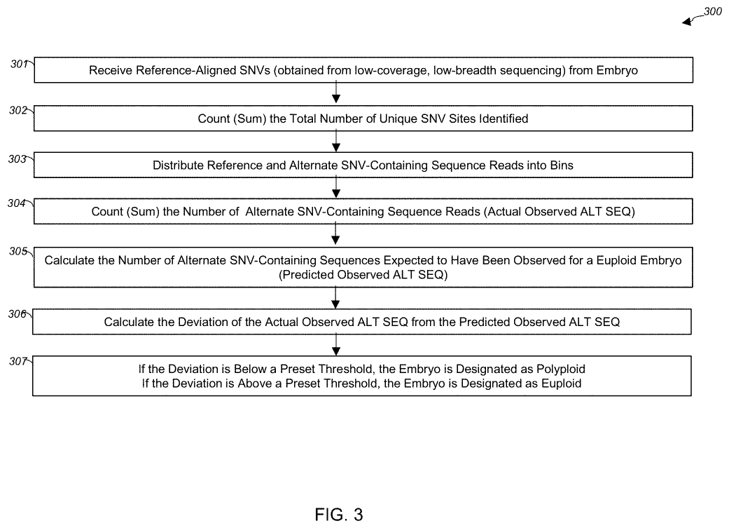

In some embodiments of the methods and systems provided herein for detecting, inferring, identifying, determining and/or distinguishing ploidy, such as polyploidy (e.g., balanced polyploidy) and/or euploidy (e.g., diploidy) and/or diploidy in a cell(s), such as, for example, an embryo, offspring and/or an organism, the difference in SNV rates of euploid and polyploid genomes is included in determining an inference of ploidy, e.g., euploidy or polyploidy (e.g., non-diploid polyploidy), such as balanced polyploidy using low-to-very low-coverage genome sequencing (e.g., such as low-coverage and/or low-depth whole genome sequencing). In such embodiments, a statistic developed based on SNV rate is used in the methods and systems that is able to detect, infer and/or determine ploidy (e.g., polyploidy) with around 90% sensitivity and specificity (see EXAMPLES herein) from low-coverage and/or low-depth, e.g., low-resolution, sequence data. FIG. 3 is a diagrammatic representation of the workflow 300 of an exemplary method provided herein.

FIG. 3 is an example diagrammatic representation of a workflow 300 of an exemplary method for detecting, inferring, identifying, determining and/or distinguishing ploidy, such as polyploidy (e.g., balanced polyploidy) and/or euploidy (e.g., diploidy), in accordance with various embodiments. As FIG. 3 illustrates one example of a method, it is understood that the combination of steps to be described can be used in various combinations as needed, with steps being removed, added or reordered. Moreover, the analysis in each step can be changed or modified per the discussion herein as needed.

As shown in of FIG. 3, reference-aligned sequence reads received in step 301 for SNVs obtained from low-coverage and/or low-depth, e.g., low-resolution, sequencing of genomic nucleic acids from an embryo are counted and summed to determine the total number of unique SNV sites identified in the sequence data.

In step 302, a total number of unique SNV sites identified are counted (or summed).

In step 303, reference and alternate SNV-containing sequence reads can be distributed into bins.

In step 304, a number of alternate SNV-containing sequence reads (Actual Observed ALT SEQ) are counted (or summed).

In step 305, a number of alternate SNV-containing sequences expected to have been observed for a euploid embryo is calculated (Predicted Observed ALT SEQ).

In step 306, the deviation of the Actual Observed ALT SEQ from the Predicted Observed ALT SEQ is calculated.

In step 307, if the deviation value is below a preset threshold, the embryo is designated as polyploid. By contrast, if the deviation is above a preset threshold, the embryo is designated as euploid.

In various embodiments, methods are provided for identifying, classifying, determining, predicting and/or inferring ploidy (e.g., monoploidy, euploidy, duploidy, balanced and unbalanced polyploidy) in an embryo. The methods can be implemented via computer software or hardware. The methods can also be implemented on a computing device/system that can include a combination of engines for identifying, classifying, determining, predicting and/or inferring polyploidy (e.g., monoploidy, euploidy, duploidy, balanced and unbalanced polyploidy) in an embryo. In various embodiments, the computing device/system can be communicatively connected to one or more of a data source, sample analyzer, and display device via a direct connection or through an internet connection.

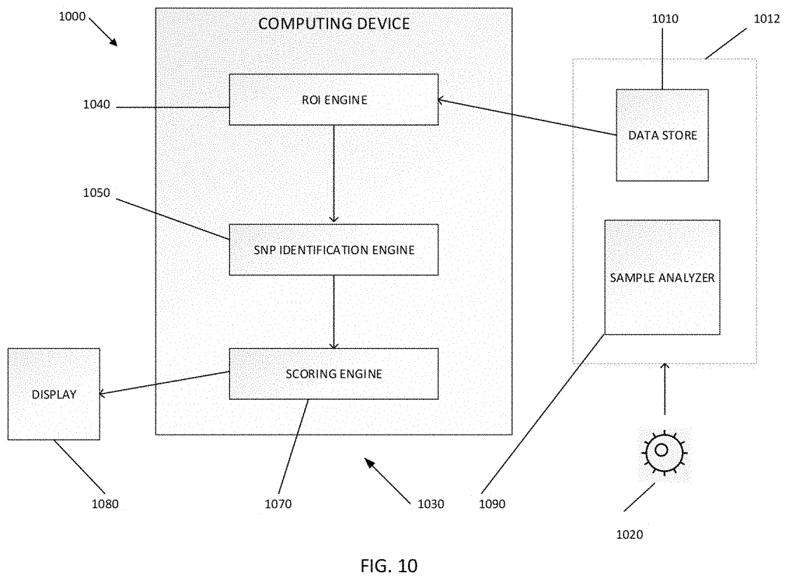

FIG. 10 is a schematic diagram of a system 1000 for detecting ploidy in an embryo (e.g., a human embryo), in accordance with various embodiments. System 1000 can include a data store 1010, a computing device 1030 and a display 1080. System 1000 can also include a sample analyzer 1090.

The sample analyzer 1090 can be communicatively connected to the data store 1010 by way of a serial bus (if both form an integrated instrument platform 1012) or by way of a network connection (if both are distributed/separate devices). The sample analyzer 1090 can be configured to analyze samples from an embryo 1020. Sample analyzer 1090 can be a sequencing instrument, such as a next generation sequencing instrument, configured to sequence samples to collect sequencing data for further analysis. In various embodiments, the sequencing data can then be stored in the data store 1010 for subsequent processing. In various embodiments, the sequencing datasets can be fed to the computing device 1030 in real-time. In various embodiments, the sequencing datasets can also be stored in the data store 1010 prior to processing. In various embodiments, the sequencing datasets can also be fed to the computing device 1030 in real-time.

The data store 1010 can be communicatively connected to the computing device 1030. In various embodiments, the computing device 1030 can be communicatively connected to the data store 1010 via a network connection that can be either a “hardwired” physical network connection (e.g., Internet, LAN, WAN, VPN, etc.) or a wireless network connection (e.g., Wi-Fi, WLAN, etc.). In various embodiments, the computing device 1030 can be a workstation, mainframe computer, distributed computing node (part of a “cloud computing” or distributed networking system), personal computer, mobile device, etc.

Data store 1010 can be configured to receive embryo sequence data. In various embodiments the embryo sequence data is acquired by low-coverage sequencing. The low-coverage sequencing can be between about 0.001 and 10×. The low-coverage sequencing can be between about 0.01 and 0.5×. The low-coverage sequencing can be between about 0.25 and 0.2×.

Computing device 1030 can further include a region of interest engine (ROI engine) 1040, a single nucleotide polymorphism identification engine (SNP identification engine) 1050, and a scoring engine 1070. As stated above, computing device 1030 can be communicatively connected to data store 1010.

ROI engine 1040 can be configured to align the received sequence data to a reference genome and identify a region of interest in the aligned embryo sequence data. The region of interest can be genome wide.

SNP identification engine 1050 can be configured to identify single nucleotide polymorphisms (SNPs) in the sequence data by comparing the received sequence data to the aligned reference genome. SNP identification engine 1050 can be further configured to filter at the embryo sequencing data to remove sequencing artifacts. The filtering can comprise excluding SNPs that are not included in a reference database of known SNPs. The reference database can include about 1000 known genomes.

Scoring engine 1070 can be configured to determine a polyploid score comprising counting the number of observed SNPs in the region of interest. Scoring engine 1070 can be configured to compare the polyploid score to a predetermined threshold. Scoring engine 1070 can be configured to identify the embryo as polyploid if the polyploid score is below the predetermined threshold. In various embodiments, the polyploid is a balanced polyploid.

After the ploidy of the embryo has been identified, a display communicatively connected to the computing device can be configured to display a report containing the polyploid classification of the embryo. It can be displayed as a result or summary on a display or client terminal 1080 that is communicatively connected to the computing device 1030. In various embodiments, display 1080 can be a thin client computing device. In various embodiments, display 1080 can be a personal computing device having a web browser (e.g., INTERNET EXPLORER™, FIREFOX™, SAFARI™, etc.) that can be used to control the operation of the region of interest engine (ROI engine) 1040, the single nucleotide polymorphism identification engine (SNP identification engine) 1050, and the scoring engine 1070.

Scoring engine 1070 can be further configured to identify the embryo as euploid if the polyploid score is above the predetermined threshold. Moreover, display 1080 can be further configured to display a report containing the euploid classification of the embryo.

It should be appreciated that the various engines can be combined or collapsed into a single engine, component or module, depending on the requirements of the particular application or system architecture. In various embodiments the region of interest engine (ROI engine) 1040, the single nucleotide polymorphism identification engine (SNP identification engine) 1050, and the scoring engine 1070 can comprise additional engines or components as needed by the particular application or system architecture.

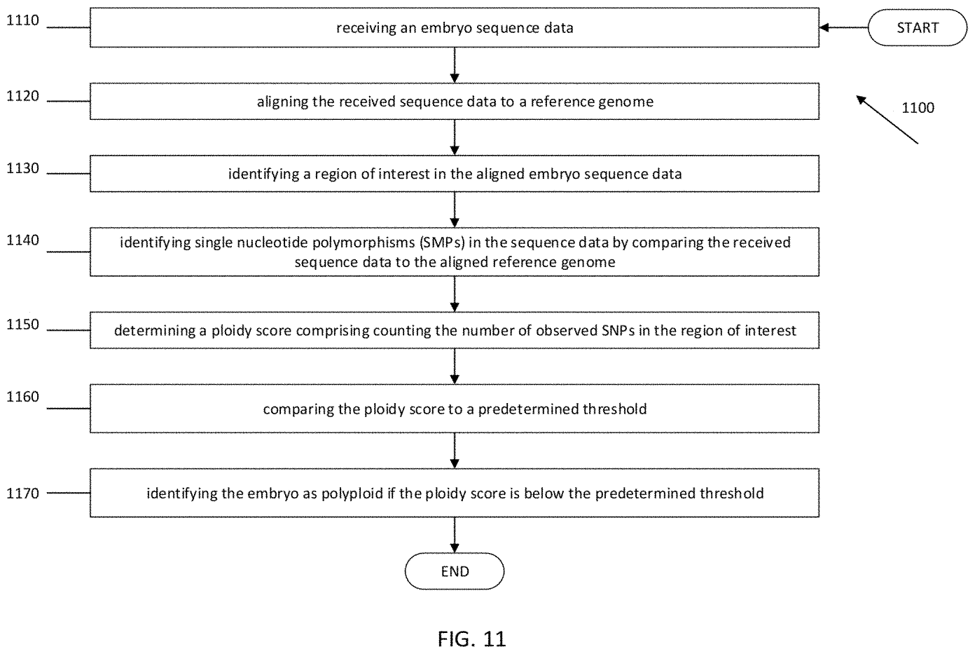

FIG. 11 is an exemplary flowchart showing a method 1100 for detecting ploidy in an embryo, in accordance with various embodiments.

In step 1110, embryo sequence data is received. In various embodiments, the embryo, sequence data is acquired by low-coverage sequencing. The low-coverage sequencing can be between about 0.001 and 10×. The low-coverage sequencing can be between about 0.01 and 0.5×. The low-coverage sequencing can be between about 0.25 and 0.2×.

In step 1120, the received sequence data is aligned to a reference genome.

In step 1130, a region of interest in the aligned embryo sequence data is identified. The region of interest can be genome wide.

In step 1140, single nucleotide polymorphisms (SNPs) in the sequence data is identified by comparing the received sequence data to the aligned reference genome. In various embodiments, the method can further comprise filtering the embryo sequencing data to remove sequencing artifacts. The filtering can comprise excluding SNPs that are not included in a reference database of known SNPs. The reference database can include about 1000 known genomes.

In step 1150, a ploidy score is determined, the score comprising counting the number of observed SNPs in the region of interest.

In step 1160, the ploidy score is compared to a predetermined threshold.

In step 1170, the embryo is identified as polyploid if the ploidy score is below the predetermined threshold. In various embodiments, the polyploid is a balanced polyploid. In various embodiments, the embryo is identified as if the ploidy score is above the predetermined threshold.

### EXAMPLES

In general, based on various embodiments disclosed herein, the expected total number of SNV occurrences observed (such as the frequency an SNV is detected) in low-to-very low-coverage NGS data is lower for the data from sequencing of polyploid genomic nucleic acids than it is for the data from sequencing of euploid genomic nucleic acids. In developing methods and systems for inferring or classifying ploidy of a genome using the variant alleles (SNV) detected in sequencing (e.g., low-coverage sequencing) of genomic nucleic acids, a ploidy variant allele detection model was established and tested. As described in these EXAMPLES, an algorithm taking into account probabilities of detecting alternate alleles in euploid and polyploid genomes in sequence information from genomic nucleic acid sequencing and factoring in sequence coverage (denoted as “depth”) was developed and improved using machine learning with sample data to build the ploidy variant allele detection model. Through this model, a prediction score was determined that can be assigned to a genomic nucleic acid sample (e.g., from an embryo) based on SNV sequence data for the sample. A threshold prediction score value was also determined. By comparing the prediction score assigned to a genomic nucleic acid sample to the threshold score, the ploidy of the sample is inferred with scores below the threshold being indicative of polyploidy.

To validate the method and observations, three flow-cells were generated of 2×36 paired-end NextSeq (IIlumina) data arrayed at 96-plex, which targets 4 million read pairs per embryo trophectoderm biopsy sample resulting in a typical coverage of about 0.1× (calculated as 4×10\hat{ }6 reads*2*36/3×10\hat{ }9 where the denominator is the genome size in base pairs and a factor of 2*36 is included in the numerator due to the paired-end sequencing (i.e., 2 reads per sequence). The data-set contains 87 human embryo cell samples of known ploidy with replicates spread across the three batches with 40 diploid cases (46:XX or 46:XY) and 10 polyploid cases (69:XXX, 69:XXY, or 96:XXXX). Data from a comma separated file was read with sample meta-data as well as genome-wide (chromosomes 1-22) digital SNV counts and, to ensure consistency of results, random number seed was set to an arbitrary value of 0. Samples with fewer than 4000000 read pairs were excluded from the analysis as were samples that were detected as having mosaic or full aneuploidy as determined by PGTai (see, e.g., described in U.S. Patent Application Publication No. 2020/0111573). The data were randomly divided into training (70% of data) and test (30% of data) sets by stratifying over replicate and polyploid class.

The training set was evaluated with an ANCOVA linear model to estimate relationship between sequencing coverage, polyploid class, and other explanatory variables. In this case, the number of heterogeneous positions (referred to as digital_count_hets) and the proportion of sequences from the original sequence file (in FASTQ) that uniquely aligned to the HG19 reference genome (rqc) and sequencing coverage (in terms of the number of read pairs aligning to reference) were input into the method.

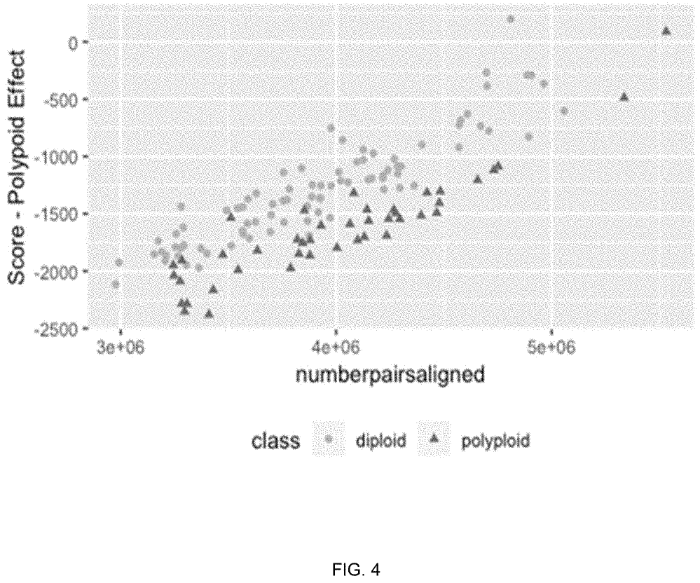

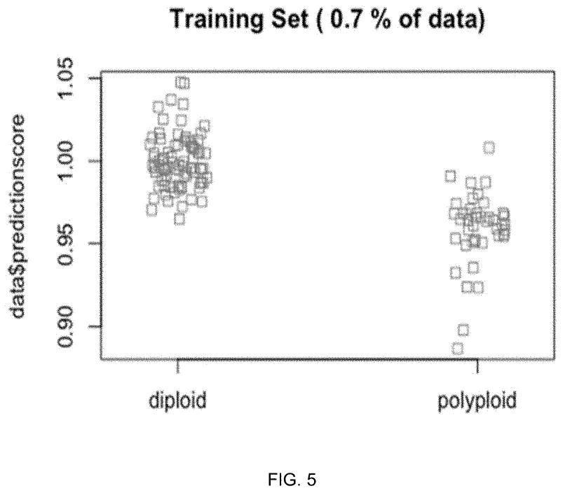

FIG. 4 presents the results of applying an algorithm corresponding to the work flow depicted in FIG. 3 to the training data set of the SNV sequencing measurements (e.g., total number of SNV sites identified, total number of sequence count for ALT alleles, total number of aligned sequence reads). as a graph of polyploid effect score vs. the number of read pairs that aligned for a sample. Each circle or triangle on the graph represents an embryo sample that was analyzed. The circles correspond to known diploid samples and the triangles correspond to known polyploid samples. This plot reflects for each sample the number of sequence read pairs from sequencing of the nucleic acids in the sample that were aligned with the reference genome (a measure of sequencing coverage). The display shown in FIG. 4 illustrates the separation obtained between the diploid and polyploid samples based on the polypoid effect score calculated in the algorithm as applied to the training data set in terms of sequencing coverage. The polyploid effect scores for each sample shown in FIG. 4 were then adjusted for the effect of sequencing coverage and other covariates to obtain a prediction score for each sample. The prediction scores for each sample are graphically indicated in FIG. 5 by aligning a square representing each sample to a point on a vertical line demarcated by increasing score. Squares lined up on the left side of the figure and labeled “diploid” below the line-up, represent diploid samples and squares lined up on the right side of the figure and labeled “polyploid” below the line-up, represent polyploid samples. FIG. 5 illustrates the separation between the polyploid classes achieved based on prediction score with most of the diploid samples having a score greater than about 0.98 and most of the polyploid samples having a score less than about 0.98.

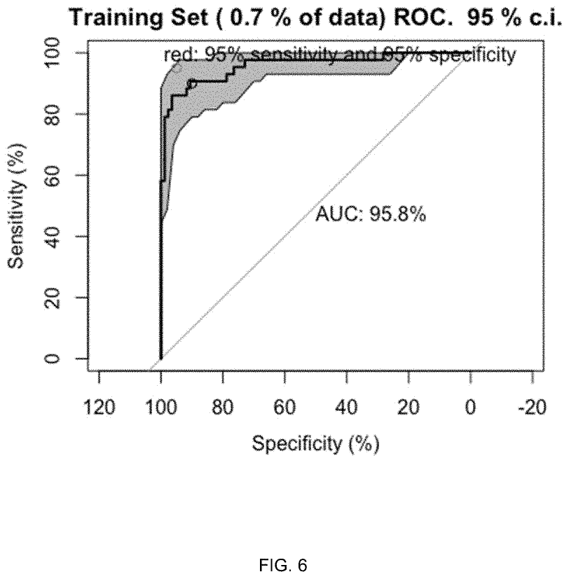

FIG. 6 illustrates a receiver operating characteristic (ROC) curve to evaluate the performance of the analysis of the training set data. The curve provides a unified display of accuracy (sensitivity and specificity) for a binary hypothesis (i.e., euploidy or polyploidy) as critical value (threshold) is raised. An optimal critical value for the threshold of c=0.9804734 is estimated from training data (Youden, 1950; to maximize distance from the diagonal line) and training set sensitivity/specificity using c is 0.91/0.91. Sensitivity 0.95 level confidence interval is estimated by 2000 bootstrapping replicates to be (0.79, 0.98). The AUC (area under the curve) value of 95.8% is a measure of the high accuracy of the method in distinguishing euploidy and polyploidy.

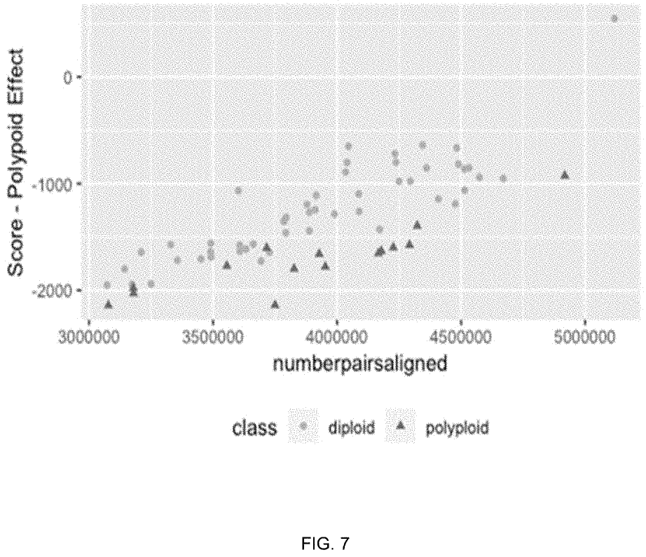

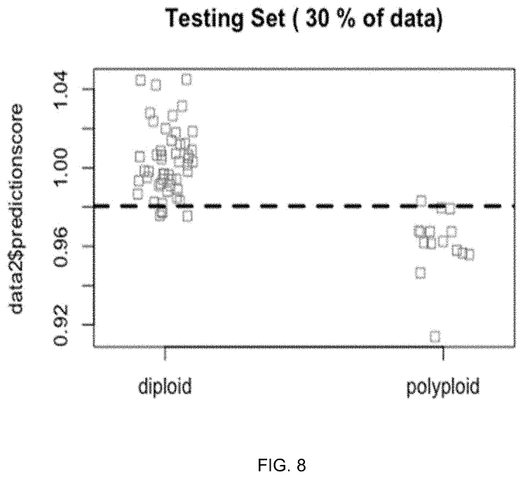

The remaining 30% of the data in the training set was then evaluated using the ploidy variant allele detection model and the critical value constructed from the training set. FIG. 7 presents the results of applying an algorithm corresponding to the work flow depicted in FIG. 3 to the training data set of the SNV sequencing measurements (e.g., total number of SNV sites identified, total number of sequence count for ALT alleles, total number of aligned sequence reads) as a graph of polyploid effect score vs. the number of read pairs that aligned for a sample. Each circle or triangle on the graph represents an embryo sample that was analyzed. The circles correspond to known diploid samples and the triangles correspond to known polyploid samples. This plot reflects for each sample the number of sequence read pairs from sequencing of the nucleic acids in the sample that were aligned with the reference genome (a measure of sequencing coverage). The display shown in FIG. 7 illustrates the separation obtained between the diploid and polyploid samples based on the polypoid effect score calculated in the algorithm as applied to the training data set in terms of sequencing coverage. The polyploid effect scores for each sample shown in FIG. 7 were then adjusted for the effect of sequencing coverage and other covariates to obtain a prediction score for each sample. The prediction scores for each sample are graphically indicated in FIG. 8 by aligning a square representing each sample to a point on a vertical line demarcated by increasing score. Squares lined up on the left side of the figure and labeled “diploid” below the line-up, represent diploid samples and squares lined up on the right side of the figure and labeled “polyploid” below the line-up, represent polyploid samples. FIG. 8 illustrates the separation between the polyploid classes achieved based on prediction score with most of the diploid samples having a score greater than about 0.98 and most of the polyploid samples having a score less than about 0.98. The horizontal line shows the threshold c=(critical value constructed from training data) and testing set sensitivity/specificity using c is estimated to be 0.93/0.92.

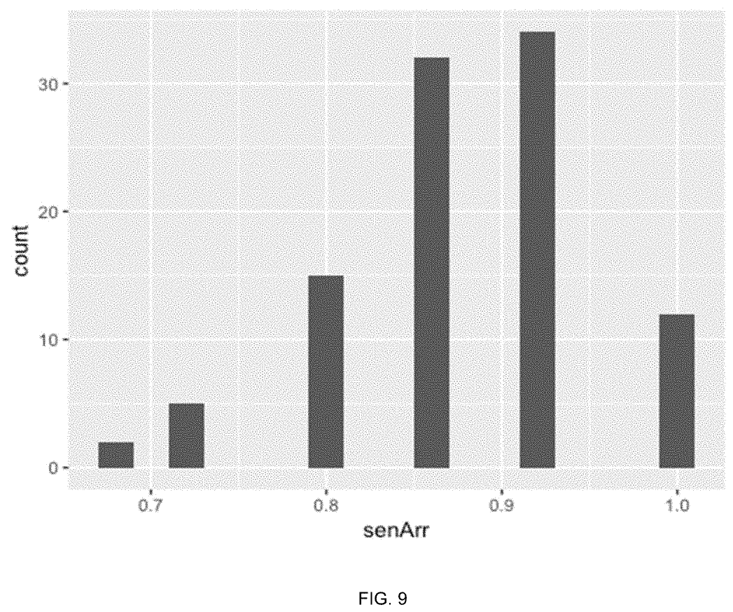

Cross validation can then be performed to further assess generality to independent datasets and to guard against possible overfitting or bias in sample selection. A 100-fold Monte Carlo cross-validation was performed where each fold entailed a procedure identical to above with stratified random sampling to split samples into training (70% of the samples) and test (30%) were used for training. As illustrated in FIG. 9, the median sensitivity/specificity measured in the test sets was 0.87/0.94 and the 95% confidence interval of sensitivity is estimated to be (0.73, 1) which is concordant with the c.i. estimated above. Best seed was 19.

### Computer Implemented System

In various embodiments, the methods for detecting ploidy in an embryo can be implemented via computer software or hardware. That is, as depicted in FIG. 10, the methods disclosed herein can be implemented on a computing device 1030 that includes a region of interest engine (ROI engine) 1040, a single nucleotide polymorphism identification engine (SNP identification engine) 1050, and a scoring engine 1070. In various embodiments, the computing device 1030 can be communicatively connected to a data store 1010 and a display device 1080 via a direct connection or through an internet connection.

It should be appreciated that the various engines depicted in FIG. 10 can be combined or collapsed into a single engine, component or module, depending on the requirements of the particular application or system architecture. Moreover, in various embodiments, the region of interest engine (ROI engine) 1040, the single nucleotide polymorphism identification engine (SNP identification engine) 1050, and the scoring engine 1070 can comprise additional engines or components as needed by the particular application or system architecture.

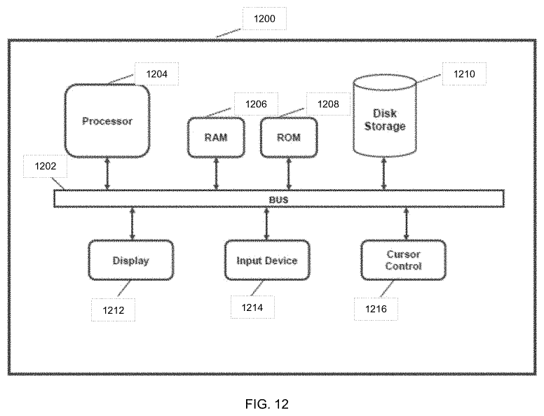

FIG. 12 is a block diagram that illustrates a computer system 1200, upon which embodiments of the present teachings may be implemented. In various embodiments of the present teachings, computer system 1200 can include a bus 1202 or other communication mechanism for communicating information, and a processor 1204 coupled with bus 1202 for processing information. In various embodiments, computer system 1200 can also include a memory, which can be a random-access memory (RAM) 1206 or other dynamic storage device, coupled to bus 1202 for determining instructions to be executed by processor 1204. Memory also can be used for storing temporary variables or other intermediate information during execution of instructions to be executed by processor 1204. In various embodiments, computer system 1200 can further include a read only memory (ROM) 1208 or other static storage device coupled to bus 1202 for storing static information and instructions for processor 1204. A storage device 1210, such as a magnetic disk or optical disk, can be provided and coupled to bus 1202 for storing information and instructions.

In various embodiments, computer system 1200 can be coupled via bus 1202 to a display 1212, such as a cathode ray tube (CRT) or liquid crystal display (LCD), for displaying information to a computer user. An input device 1214, including alphanumeric and other keys, can be coupled to bus 1202 for communicating information and command selections to processor 1204. Another type of user input device is a cursor control 1216, such as a mouse, a trackball or cursor direction keys for communicating direction information and command selections to processor 1204 and for controlling cursor movement on display 1212. This input device 1214 typically has two degrees of freedom in two axes, a first axis (i.e., x) and a second axis (i.e., y), that allows the device to specify positions in a plane. However, it should be understood that input devices 1214 allowing for 3-dimensional (x, y and z) cursor movement are also contemplated herein.

Consistent with certain implementations of the present teachings, results can be provided by computer system 1200 in response to processor 1204 executing one or more sequences of one or more instructions contained in memory 1206. Such instructions can be read into memory 1206 from another computer-readable medium or computer-readable storage medium, such as storage device 1210. Execution of the sequences of instructions contained in memory 1206 can cause processor 1204 to perform the processes described herein. Alternatively, hard-wired circuitry can be used in place of or in combination with software instructions to implement the present teachings. Thus, implementations of the present teachings are not limited to any specific combination of hardware circuitry and software.

The term “computer-readable medium” (e.g., data store, data storage, etc.) or “computer-readable storage medium” as used herein refers to any media that participates in providing instructions to processor 1204 for execution. Such a medium can take many forms, including but not limited to, non-volatile media, volatile media, and transmission media. Examples of non-volatile media can include, but are not limited to, optical, solid state, magnetic disks, such as storage device 1210. Examples of volatile media can include, but are not limited to, dynamic memory, such as memory 1206. Examples of transmission media can include, but are not limited to, coaxial cables, copper wire, and fiber optics, including the wires that comprise bus 1202.

Common forms of computer-readable media include, for example, a floppy disk, a flexible disk, hard disk, magnetic tape, or any other magnetic medium, a CD-ROM, any other optical medium, punch cards, paper tape, any other physical medium with patterns of holes, a RAM, PROM, and EPROM, a FLASH-EPROM, any other memory chip or cartridge, or any other tangible medium from which a computer can read.

In addition to computer readable medium, instructions or data can be provided as signals on transmission media included in a communications apparatus or system to provide sequences of one or more instructions to processor 1204 of computer system 1200 for execution. For example, a communication apparatus may include a transceiver having signals indicative of instructions and data. The instructions and data are configured to cause one or more processors to implement the functions outlined in the disclosure herein. Representative examples of data communications transmission connections can include, but are not limited to, telephone modem connections, wide area networks (WAN), local area networks (LAN), infrared data connections, NFC connections, etc.

It should be appreciated that the methodologies described herein flow charts, diagrams and accompanying disclosure can be implemented using computer system 1200 as a standalone device or on a distributed network of shared computer processing resources such as a cloud computing network.

The methodologies described herein may be implemented by various means depending upon the application. For example, these methodologies may be implemented in hardware, firmware, software, or any combination thereof. For a hardware implementation, the processing unit may be implemented within one or more application specific integrated circuits (ASICs), digital signal processors (DSPs), digital signal processing devices (DSPDs), programmable logic devices (PLDs), field programmable gate arrays (FPGAs), processors, controllers, micro-controllers, microprocessors, electronic devices, other electronic units designed to perform the functions described herein, or a combination thereof.

In various embodiments, the methods of the present teachings may be implemented as firmware and/or a software program and applications written in conventional programming languages such as C, C++, Python, etc. If implemented as firmware and/or software, the embodiments described herein can be implemented on a non-transitory computer-readable medium in which a program is stored for causing a computer to perform the methods described above. It should be understood that the various engines described herein can be provided on a computer system, such as computer system 1200 of FIG. 12, whereby processor 1204 would execute the analyses and determinations provided by these engines, subject to instructions provided by any one of, or a combination of, memory components 1206/1208/1210 and user input provided via input device 1214.

While the present teachings are described in conjunction with various embodiments, it is not intended that the present teachings be limited to such embodiments. On the contrary, the present teachings encompass various alternatives, modifications, and equivalents, as will be appreciated by those of skill in the art.

In describing various embodiments, the specification may have presented a method and/or process as a particular sequence of steps. However, to the extent that the method or process does not rely on the particular order of steps set forth herein, the method or process should not be limited to the particular sequence of steps described. As one of ordinary skill in the art would appreciate, other sequences of steps may be possible. Therefore, the particular order of the steps set forth in the specification should not be construed as limitations on any claims. In addition, any claims directed to the method and/or process should not be limited to the performance of their steps in the order written, and one skilled in the art can readily appreciate that the sequences may be varied and still remain within the spirit and scope of the various embodiments.

### RECITATION OF EMBODIMENTS

Embodiment 1: A method for detecting ploidy in an embryo, comprising:
receiving an embryo sequence data;
aligning the received sequence data to a reference genome;
identifying a region of interest in the aligned embryo sequence data;
identifying single nucleotide polymorphisms (SMPs) in the sequence data by comparing the received sequence data to the aligned reference genome;
determining a ploidy score comprising counting the number of observed SNPs in the region of interest;
comparing the ploidy score to a predetermined threshold; and
identifying the embryo as polyploid if the ploidy score is below the predetermined threshold.

Embodiment 2: The method of Embodiment 1, further comprising identifying the embryo as euploid if the ploidy score is above the predetermined threshold.

Embodiment 3: The method of Embodiments 1 or 2, wherein the polyploid is a balanced polyploid.

Embodiment 4: The method of any one of Embodiments 1 to 3, wherein the embryo sequence data is acquired by low-coverage sequencing.

Embodiment 5: The method of Embodiment 4, wherein the low-coverage sequencing is between about 0.001 and 10×.

Embodiment 6: The method of Embodiment 4, wherein the low-coverage sequencing is between about 0.01 and 0.5×.

Embodiment 7: The method of Embodiment 4, wherein the low-coverage sequencing is between about 0.25 and 0.2×.

Embodiment 8: The method of any one of Embodiments 1 to 7, wherein the region of interest is genome wide.

Embodiment 9: The method of any one of Embodiments 1 to 8, further comprising filtering the embryo sequencing data to remove sequencing artifacts.

Embodiment 10: The method of Embodiment 9, wherein the filtering comprises excluding SNPs that are not included in a reference database of known SNPs.

Embodiment 11: The method of Embodiment 10, wherein the reference database includes about 1000 known genomes.

Embodiment 12: A non-transitory computer-readable medium storing computer instructions for detecting ploidy in an embryo, comprising:
receiving an embryo sequence data;
aligning the received sequence data to a reference genome;
identifying a region of interest in the aligned embryo sequence data;
identifying single nucleotide polymorphisms (SMPs) in the sequence data by comparing the received sequence data to the aligned reference genome;
determining a ploidy score comprising counting the number of observed SNPs in the region of interest;
comparing the ploidy score to a predetermined threshold; and
identifying the embryo as polyploid if the ploidy score is below the predetermined threshold.

Embodiment 13: The method of Embodiment 12, further comprising identifying the embryo as euploid if the ploidy score is above the predetermined threshold.

Embodiment 14: The method of Embodiments 12 or 13, wherein the polyploid is a balanced polyploid.

Embodiment 15: The method of any one of Embodiments 12 to 14, wherein the embryo sequence data is acquired by low-coverage sequencing.

Embodiment 16: The method of Embodiment 15, wherein the low-coverage sequencing is between about 0.001 and 10×.

Embodiment 17: The method of Embodiment 15, wherein the low-coverage sequencing is between about 0.01 and 0.5×.

Embodiment 18: The method of Embodiment 15, wherein the low-coverage sequencing is between about 0.25 and 0.2×.

Embodiment 19: The method of any of Embodiments 12 to 18, wherein the region of interest is genome wide.

Embodiment 20: The method of any of the Embodiments claims 12 to 19, further comprising filtering the embryo sequencing data to remove sequencing artifacts.

Embodiment 21: The method of Embodiment 20, wherein the filtering comprises excluding SNPs that are not included in a reference database of known SNPs.

Embodiment 22: The method of Embodiment 21, wherein the reference database includes about 1000 known genomes.

Embodiment 23: A system for detecting ploidy in an embryo, comprising:
a data store for receiving an embryo sequence data;
a computing device communicatively connected to the data store, the computing device comprising
   an ROI engine configured to align the received sequence data to a reference genome, and identify a region of interest in the aligned embryo sequence data;
   a SNP identification engine configured to identify single nucleotide polymorphisms (SMPs) in the sequence data by comparing the received sequence data to the aligned reference genome; and
   a scoring engine configured to determine a polyploid score comprising counting the number of observed SNPs in the region of interest, compare the polyploid score to a predetermined threshold, and identifying the embryo as polyploid if the polyploid score is below the predetermined threshold;
and
a display communicatively connected to the computing device and configured to display a report containing the polyploid classification of the embryo.

Embodiment 24: The system of Embodiment 23, wherein the scoring engine is further configured to identify the embryo as euploid if the polyploid score is above the predetermined threshold.

Embodiment 25: The system of Embodiments 23 or 24, wherein the display is further configured to display a report containing the euploid classification of the embryo.

Embodiment 26: The system of any of Embodiments 23 to 25, wherein the polyploid is a balanced polyploid.

Embodiment 27: The system of any of Embodiments 23 to 26, wherein the embryo sequence data is acquired by low-coverage sequencing.

Embodiment 28: The system of Embodiment 27, wherein the low-coverage sequencing is between about 0.001 and 10×.

Embodiment 29: The system of Embodiment 27, wherein the low-coverage sequencing is between about 0.01 and 0.5×.

Embodiment 30: The system of Embodiment 27, wherein the low-coverage sequencing is between about 0.25 and 0.2×.

Embodiment 31: The system of any of Embodiments 23 to 30, wherein the region of interest is genome wide.

Embodiment 32: The system of any of Embodiments 23 to 31, wherein the SNP identification engine is further configured to filter the embryo sequencing data to remove sequencing artifacts.

Embodiment 33: The system of Embodiment 32, wherein the filtering comprises excluding SNPs that are not included in a reference database of known SNPs.

Embodiment 34: The system of Embodiment 33, wherein the reference database includes about 1000 known genomes.

1. A method for detecting ploidy in an embryo, comprising:
receiving an embryo sequence data;
aligning the received sequence data to a reference genome;
identifying a region of interest in the aligned embryo sequence data;
identifying single nucleotide polymorphisms (SMPs) in the sequence data by comparing the received sequence data to the aligned reference genome;
determining a ploidy score comprising counting the number of observed SNPs in the region of interest;
comparing the ploidy score to a predetermined threshold; and
identifying the embryo as polyploid if the ploidy score is below the predetermined threshold.

2. The method of claim 1, further comprising identifying the embryo as euploid if the ploidy score is above the predetermined threshold.

3. The method of claim 1, wherein the polyploid is a balanced polyploid.

4. The method of claim 1, wherein the embryo sequence data is acquired by low-coverage sequencing.

5. The method of claim 4, wherein the low-coverage sequencing is between about 0.001 and 10×.

6. The method of claim 4, wherein the low-coverage sequencing is between about 0.01 and 0.5×.

7. The method of claim 4, wherein the low-coverage sequencing is between about 0.25 and 0.2×.

8. (canceled)

9. (canceled)

10. (canceled)

11. (canceled)

12. A non-transitory computer-readable medium storing computer instructions for detecting ploidy in an embryo, comprising:
receiving an embryo sequence data;
aligning the received sequence data to a reference genome;
identifying a region of interest in the aligned embryo sequence data;
identifying single nucleotide polymorphisms (SMPs) in the sequence data by comparing the received sequence data to the aligned reference genome;
determining a ploidy score comprising counting the number of observed SNPs in the region of interest;
comparing the ploidy score to a predetermined threshold; and
identifying the embryo as polyploid if the ploidy score is below the predetermined threshold.

13. The method of claim 12, further comprising identifying the embryo as euploid if the ploidy score is above the predetermined threshold.

14. The method of claim 13, wherein the polyploid is a balanced polyploid.

15. The method of claim 12, wherein the embryo sequence data is acquired by low-coverage sequencing.

16. The method of claim 15, wherein the low-coverage sequencing is between about 0.001 and 10×.

17. The method of claim 15, wherein the low-coverage sequencing is between about 0.01 and 0.5×.

18. The method of claim 15, wherein the low-coverage sequencing is between about 0.25 and 0.2×.

19. (canceled)

20. (canceled)

21. (canceled)

22. (canceled)

23. A system for detecting ploidy in an embryo, comprising:
a data store for receiving an embryo sequence data;
a computing device communicatively connected to the data store, the computing device comprising
an ROI engine configured to align the received sequence data to a reference genome, and identify a region of interest in the aligned embryo sequence data;
a SNP identification engine configured to identify single nucleotide polymorphisms (SMPs) in the sequence data by comparing the received sequence data to the aligned reference genome; and
a scoring engine configured to determine a polyploid score comprising counting the number of observed SNPs in the region of interest, compare the polyploid score to a predetermined threshold, and identifying the embryo as polyploid if the polyploid score is below the predetermined threshold;
and
a display communicatively connected to the computing device and configured to display a report containing the polyploid classification of the embryo.

24. The system of claim 23, wherein the scoring engine is further configured to identify the embryo as euploid if the polyploid score is above the predetermined threshold.

25. (canceled)

26. The system of claim 23, wherein the polyploid is a balanced polyploid.

27. The system of claim 23, wherein the embryo sequence data is acquired by low-coverage sequencing.

28. The system of claim 27, wherein the low-coverage sequencing is between about 0.001 and 10×.

29. The system of claim 27, wherein the low-coverage sequencing is between about 0.01 and 0.5×.

30. (canceled)

31. (canceled)

32. (canceled)

33. (canceled)

34. (canceled)
